(19) United States
(12) Patent Application Publication
(10) Pub. No.: US 2022/0002915 A1
(43) Pub. Date: Jan. 6, 2022

(54) WEARABLE SENSOR, FORMING METHOD THEREFOR, AND SENSOR MODULE

(71) Applicant: MEDX TECHNOLOGY INC., Taipei City (TW)

(72) Inventor: Yi-Yuan Chen, Taipei (TW)

(73) Assignee: MEDX TECHNOLOGY INC., Taipei City (TW)

(21) Appl. No.: 17/284,752

(22) PCT Filed: Oct. 10, 2019
(86) PCT No.: PCT/CN2019/110310

§ 371 Date: Apr. 12, 2021

(30) Foreign Application Priority Data
Oct. 12, 2018 (CN) 201811191250.2

(51) Int. Cl. D02G 3/44; D03D 1/00; A61B 5/00

(52) U.S. Cl. CPC D02G 3/441; D03D 1/0088; D10B 2403/02431; D10B 2401/16; A61B 5/6802

(57) ABSTRACT

A wearable sensor, a forming method therefor, and a sensor module. The wearable sensor includes elastic yarns made of an elastic material; and conductive yarns. The conductive yarns and the elastic yarns interweave and form a fabric structure. The conductive yarn has a first end, a second end, and a body portion between the first end and the second end, the body portion include an entry section extending from the first end toward a fold-back region and an exit section returned back from the fold-back region, the entry section and the exit section form at least one intersection, and the entry section and the exit section come into contact at the intersection. In addition to providing the sensing measurement function, said sensor can improve the user experience, and can expand the application field of the sensor.

### TECHNICAL FIELD

The present invention relates to the technical field of electrics, and particularly relates to a wearable sensor, a forming method thereof, and a sensor module.

### BACKGROUND

With the development of technology, wearable sensing equipment is getting more and more popular. For example, equipment such as a sports bracelet can monitor user's motion, sleep state, heart rate and other data.

The wearable sensing equipment in the prior art is generally provided with hardware equipment, such as a gyroscope, an accelerometer, a pressure sensor and a magnetometer.

However, the existing sensing equipment requires a user to wear additionally and is not comfortable enough to wear, which brings inconvenience to the user's use; and the part subject to sensing measurement and its application are limited.

### SUMMARY

The technical problem to be solved by the present invention is how to improve the use convenience of a wearable sensor.

In order to solve the above technical problem, one or more embodiments of the present invention provide a wearable sensor, including: an elastic yarn made of an elastic material; and a conductive yarn having a conductive capability; wherein the conductive yarn and the elastic yarn interweave to form a fabric structure; wherein the conductive yarn has a first end, a second end, and a body portion between the first end and the second end, the body portion includes an entry section extending from the first end toward a fold-back region and an exit section returned back from the fold-back region, the entry section and the exit section form at least one intersection, and the entry section and the exit section come into contact at the intersection.

In one or more embodiments, an area formed by the contact of the entry section and the exit section at the intersection changes with the elastic deformation of the elastic yarn.

In one or more embodiments, when the fabric structure is deformed due to an external force, the area formed by the contact at the intersection increases with the elastic deformation of the elastic yarn.

In one or more embodiments, when the fabric structure is deformed due to an external force, the area formed by the contact at the intersection decreases with the elastic deformation of the elastic yarn.

In one or more embodiments, there may be a plurality of the conductive yarns, and the plurality of conductive yarns are twined or not twined with each other.

In one or more embodiments, the entry section includes a plurality of adjacent first sub-sections; the exit section includes a plurality of adjacent second sub-sections; wherein the plurality of adjacent first sub-sections and the plurality of adjacent second sub-sections intersect and come into contact at the intersections.

In one or more embodiments, one of the entry section and the exit section includes a straight section extending along a straight line, and the other one includes a plurality of return sub-sections and connecting sub-sections connecting the return sub-sections and the straight section; wherein the return sub-sections and the straight section form intersections and come into contact at the intersections.

In one or more embodiments, the conductive yarn and the elastic yarn interweave by a fabric weaving method; wherein the exit section intersects with the entry section in a secondary fold-back region and then extends toward the fold-back region to form a secondary entry section, and the secondary entry section intersects with the exit section in the fold-back region and then folds back to form a secondary exit section.

In one or more embodiments, the secondary exit section intersects with the secondary entry section in the secondary fold-back region and then extends toward the fold-back region again.

In one or more embodiments, the conductive yarn is made of copper, silver, stainless steel, or other metallic materials with high conductive coefficients.

In one or more embodiments, the conductive yarn has a wrapping structure.

In one or more embodiments, the wrapping structure includes a central thread and a covering thread twined outside the central thread; wherein the central thread is made of a conductive material and the covering thread is made of a non-conductive material, or the central thread is made of a non-conductive material and the covering thread is made of a conductive material.

In order to solve the above technical problem, one or more embodiments of the present invention further disclose a forming method of the wearable sensor, the forming method of the sensor including: providing an elastic yarn made of an elastic material; providing a conductive yarn having a conductive capability, the conductive yarn having a first end, a second end, and a body portion between the first end and the second end; and interweaving the conductive yarn and the elastic yarn to form a fabric structure, wherein the fabric structure has a fold-back region, a part of the body portion that extends from the first end toward the fold-back region is used as an entry section, a part returned back from the fold-back region is used as an exit section, the entry section and the exit section form at least one intersection, and the entry section and the exit section come into contact at the intersection.

In one or more embodiments, an area formed by the contact of the entry section and the exit section at the intersection changes with the elastic deformation of the elastic yarn.

In one or more embodiments, when the fabric structure is deformed due to an external force, the area formed by the contact at the intersection increases with the elastic deformation of the elastic yarn.

In one or more embodiments, when the fabric structure is deformed due to an external force, the area formed by the contact at the intersection decreases with the elastic deformation of the elastic yarn.

In one or more embodiments, there are a plurality of the conductive yarns, and the plurality of conductive yarns are twined or not twined with each other.

In one or more embodiments, the entry section includes a plurality of adjacent first sub-sections; the exit section includes a plurality of adjacent second sub-sections; and interweaving the entry section and the exit section with the elastic yarn to form at least one intersection including: making the plurality of adjacent first sub-sections and the plurality of adjacent second sub-sections interweave with the elastic yarn and come into contact at the intersections.

In one or more embodiments, one of the entry section and the exit section includes a straight section extending along a straight line, and the other one includes a plurality of return sub-sections and connecting sub-sections connecting the return sub-sections and the straight section; and the return sub-sections, the straight section and the elastic yarn interweave to form intersections and come into contact at the intersections.

In one or more embodiments, the conductive yarn and the elastic yarn interweave by a fabric weaving method; the exit section intersects with the entry section in a secondary fold-back region and then extends toward the fold-back region to form a secondary entry section; and the secondary entry section intersects with the exit section in the fold-back region and then folds back to form a secondary exit section.

In one or more embodiments, the secondary exit section intersects with the secondary entry section in the secondary fold-back region and then extends toward the fold-back region again.

In one or more embodiments, the conductive yarn is made of copper, silver, stainless steel, or other metallic materials with high conductive coefficients.

In one or more embodiments, the conductive yarn has a wrapping structure.

In one or more embodiments, the wrapping structure includes a central thread and a covering thread twined outside the central thread; wherein the central thread is made of a conductive material and the covering thread is made of a non-conductive material, or the central thread is made of a non-conductive material and the covering thread is made of a conductive material.

One or more embodiments of the present invention further disclose a sensor module, including a plurality of wearable sensors; wherein in the adjacent wearable sensors, the second end of the conductive yarn of one wearable sensor is connected to the first end of the conductive yarn of the other wearable sensor.

One or more embodiments of the present invention further disclose a forming method of the sensor module, wherein, in the adjacent wearable sensors, connecting the second end of the conductive yarn of one wearable sensor to the first end of the conductive yarn of the other wearable sensor.

One or more embodiments of the present invention further disclose a wearable sensor, including: a first fabric layer having at least one conductive area; and a second fabric layer attached to the first fabric layer, the second fabric layer including: an elastic yarn made of an elastic material; and a conductive yarn having a conductive capability and having at least a wrapping structure; wherein the conductive yarn and the elastic yarn interweave to form a fabric structure; wherein the conductive yarn has a first end, a second end, and a body portion between the first end and the second end, and the body portion does not have any intersection.

In one or more embodiments, when the fabric structure is deformed due to an external force, a contact area of the second fabric layer and the first fabric layer changes with the elastic deformation of the elastic yarn.

In one or more embodiments, the wrapping structure includes a central thread and a covering thread twined outside the central thread; wherein the central thread is made of a non-conductive material and the covering thread is made of a conductive material.

In one or more embodiments, the body portion includes a plurality of adjacent U-shaped connecting sections.

In one or more embodiments, the first fabric layer includes the elastic yarns and the conductive yarn, and the elastic yarn is made of an elastic material; the conductive yarn has a conductive capability and has at least a wrapping structure; the conductive yarn and the elastic yarn interweave to form a fabric structure; wherein the conductive yarn has a first end, a second end, and a body portion between the first end and the second end, and the body portion does not have any intersection.

In one or more embodiments, the body portion of the conductive yarn in the first fabric layer and the body portion of the conductive yarn in the second fabric layer are perpendicular or parallel to each other.

In one or more embodiments, the first fabric layer includes a raised fabric portion, which is formed by controlling the interweaving tension of the elastic yarns in the first fabric layer.

One or more embodiments of the present invention further disclose a forming method of the wearable sensor, the forming method including: providing a first fabric layer having at least one conductive area; providing a second fabric layer attached to the first fabric layer, the second fabric layer including an elastic yarn made of an elastic material and a conductive yarn having a conductive capability and having at least a wrapping structure; and interweaving the conductive yarn and the elastic yarn in the second fabric layer to form a fabric structure, wherein the conductive yarn has a first end, a second end, and a body portion between the first end and the second end, and the body portion does not have any intersection.

In one or more embodiments, when the fabric structure is deformed due to an external force, a contact area of the second fabric layer and the first fabric layer changes with the elastic deformation of the elastic yarn.

In one or more embodiments, the wrapping structure includes a central thread and a covering thread twined outside the central thread; wherein the central thread is made of a non-conductive material and the covering thread is made of a conductive material.

In one or more embodiments, the body portion includes a plurality of adjacent U-shaped connecting sections.

In one or more embodiments, the first fabric layer includes the elastic yarn and the conductive yarn, and the elastic yarn is made of an elastic material; the conductive yarn has a conductive capability and has at least a wrapping structure; providing the first fabric layer includes: interweaving the conductive yarns and the elastic yarns in the first fabric layer to form a fabric structure; wherein the conductive yarn has a first end, a second end, and a body portion between the first end and the second end, and the body portion does not have any intersection.

In one or more embodiments, the body portion of the conductive yarn in the first fabric layer and the body portion of the conductive yarn in the second fabric layer are perpendicular or parallel to each other.

In one or more embodiments, the first fabric layer includes an elastic yarn, and providing the first fabric layer includes: controlling the interweaving tension of the elastic yarn in the first fabric layer to form a raised fabric portion.

Compared with the prior art, the technical solutions of one or more embodiments of the present invention have the following beneficial effects:

The wearable sensor of one or more embodiments of the present invention includes: an elastic yarn made of an elastic material; and a conductive yarn having a conductive capability; wherein the conductive yarn and the elastic yarn interweave to form a fabric structure; wherein the conductive yarn has a first end, a second end, and a body portion between the first end and the second end, the body portion includes an entry section extending from the first end toward a fold-back region and an exit section returned back from the fold-back region, the entry section and the exit section form at least one intersection, and the entry section and the exit section come into contact at the intersection. In the technical solution of the present invention, the conductive yarn and the elastic yarn interweave to form a fabric structure, and the entry section and the exit section form at least one intersection, to form impedance between the first end and the second end, so that an electrical signal may be measured between the first end and the second end to realize a sensing measurement function; in addition, because the sensor is formed by the elastic yarn and the conductive yarn, the sensor has good flexibility, improves wearing comfort, and then improves user experience.

Further, an area formed by the contact of the entry section and the exit section at the intersection changes with the elastic deformation of the elastic yarn; and when the fabric structure is deformed due to an external force, the area formed by the contact at the intersection increases or decreases with the elastic deformation of the elastic yarn. In the technical solution of the present invention, the deformation of the elastic yarn affects the change in the contact area of the intersection, and the change in the contact area of the intersection affects the change of the impedance of the conductive yarn, that is, the change in the impedance between the first end and the second end, so that when the motion of a part to be detected causes the deformation of the elastic yarn, the wearable sensor can detect the motion of the part to be detected, which increases the application range of the sensor.

### BRIEF DESCRIPTION OF DRAWINGS

FIG. 1 is a schematic structural diagram of a wearable sensor according to Embodiment 1 of the present invention;

FIG. 2 is a schematic diagram of a specific structure of a body portion shown in FIG. 1;

FIG. 3 is a schematic structural diagram of a wearable sensor according to Embodiment 2 of the present invention;

FIG. 4 is a schematic diagram of a specific structure of a body portion shown in FIG. 3;

FIG. 5 is a schematic structural diagram of a wearable sensor according to Embodiment 3 of the present invention;

FIG. 6 is a schematic structural diagram of a sensor module according to an embodiment of the present invention;

FIG. 7 is a schematic structural diagram of a wearable sensor according to Embodiment 4 of the present invention;

FIG. 8 is a schematic structural diagram of a wearable sensor according to Embodiment 5 of the present invention;

FIG. 9 is a schematic principle diagram of area change of an intersection according to an embodiment of the present invention;

FIG. 10 is a schematic structural diagram of a conductive yarn with a wrapping structure according to an embodiment of the present invention;

FIG. 11 is a schematic diagram of performance comparison of a conductive yarn with a wrapping structure and a conductive yarn without a wrapping structure.

### DETAILED DESCRIPTION

As described in the background art, the existing sensing equipment requires a user to wear additionally and is not comfortable enough to wear, which brings inconvenience to the user's use.

In one or more embodiments of the present invention, a conductive yarn and an elastic yarn interweave to form a fabric structure, and an entry section and an exit section form at least one intersection, to form impedance between a first end and a second end, so that an electrical signal may be measured between the first end and the second end to realize a sensing measurement function; in addition, because the sensor is formed by the elastic yarn and the conductive yarn, the sensor has good flexibility, improves wearing comfort, and then improves user experience.

To make the objectives, features and advantages of the present invention more obvious and understandable, specific embodiments of the present invention will be described in detail below in conjunction with the accompanying drawings.

One or more embodiments of the present invention disclose a wearable sensor, including: an elastic yarn made of an elastic material; and a conductive yarn having a conductive capability. The conductive yarn and the elastic yarn interweave to form a fabric structure.

During specific implementation, the conductive yarn and the elastic yarn interweave to form a fabric structure by a fabric weaving method, for example, the fabric structure may be cloth. The fabric weaving method may be a knitting method or a tatting method. Further, the elastic yarn can first interweave to form an elastic cloth, and then the conductive yarn is sewn to the surface of the elastic cloth, or the conductive yarn and the elastic yarn simultaneously interweave to form a fabric structure. The elastic yarn in the fabric structure has elasticity, so that the entire fabric structure has elasticity. In addition, the conductive yarn in the fabric structure has a conductive capability.

The conductive yarn has a first end, a second end, and a body portion between the first end and the second end, the body portion includes an entry section extending from the first end toward a fold-back region and an exit section returned back from the fold-back region, the entry section and the exit section form at least one intersection, and the entry section and the exit section come into contact at the intersection.

In a nonrestrictive embodiment of the present invention, an area formed by the contact of the entry section and the exit section at the intersection changes with the elastic deformation of the elastic yarn.

Further, when the fabric structure is deformed due to an external force, the area formed by the contact at the intersection increases with the elastic deformation of the elastic yarn. In one or more embodiments, when the fabric structure is deformed due to an external force, the area formed by the contact at the intersection decreases with the elastic deformation of the elastic yarn.

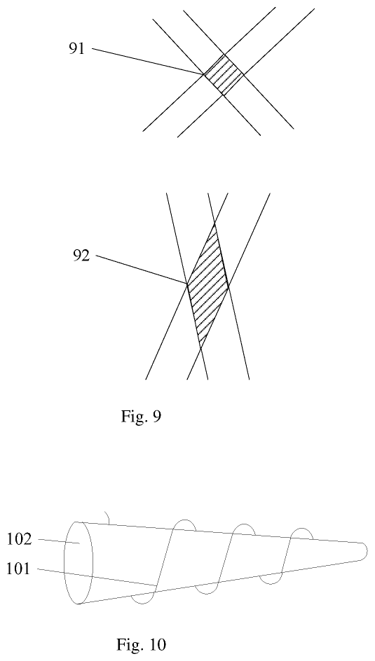

Specifically, referring to FIG. 9, before the fabric structure is stretched, the contact area formed by the intersection is shown by 91; and after the fabric structure is stretched, the contact area formed by the intersection is shown by 92, so the contact area formed by the intersection increases with the elastic deformation of the elastic yarn. In one or more embodiments, before the fabric structure is stretched, the contact area formed by the intersection is shown by 92; and after the fabric structure is stretched, the contact area formed by the intersection is shown by 91, so the contact area formed by the intersection decreases with the elastic deformation of the elastic yarn.

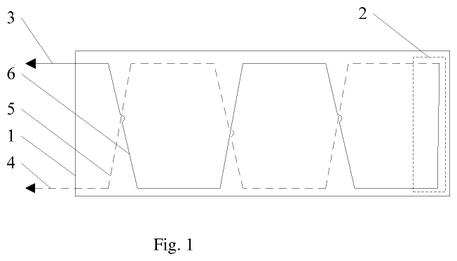

In a nonrestrictive embodiment 1 of the present invention, as shown in FIG. 1, the wearable sensor includes an elastic yarn. The elastic yarn and a conductive yarn interweave to form a fabric structure 1. A fold-back region 2 is located within the fabric structure 1.

The conductive yarn has a first end 3, a second end 4, and a body portion (not shown). The solid line shows an entry section 6 extending from the first end 3 toward the fold-back region 2; and the broken line shows an exit section 5 returned back from the fold-back region 2.

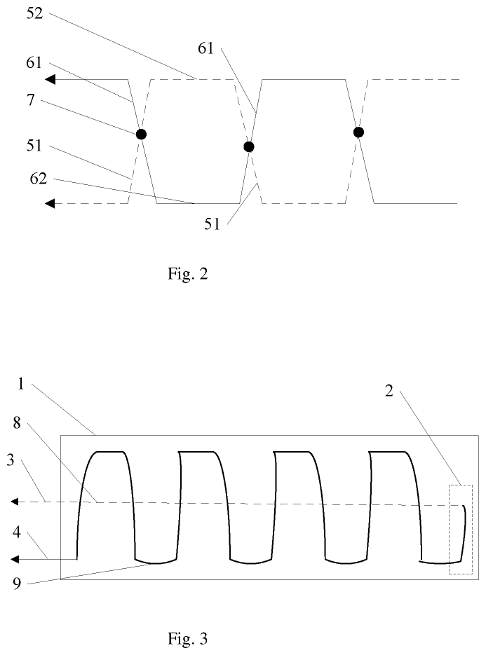

Specifically, referring to FIG. 2, the entry section 6 includes a plurality of adjacent first sub-sections (not shown); the exit section 5 includes a plurality of adjacent second sub-sections (not shown); wherein the plurality of adjacent first sub-sections and the plurality of adjacent second sub-sections intersect and come into contact at intersections 7.

More specifically, the first sub-section includes a plurality of first affiliated sub-sections 61 and first connecting sub-sections 62 connecting the adjacent first affiliated sub-sections 62, and the angles formed by the first affiliated sub-sections 61 and the first connecting sub-sections 62 are not right angles, or the angles formed by the first affiliated sub-sections 61 and the first connecting sub-sections 62 are right angles. The first connecting sub-sections 62 are straight lines or curves.

The second sub-section includes a plurality of second affiliated sub-sections 51 and second connecting sub-sections 52 connecting the adjacent second affiliated sub-sections 52, and the angles formed by the second affiliated sub-sections 51 and the second connecting sub-sections 52 are not right angles, or the angles formed by the second affiliated sub-sections 51 and the second connecting sub-sections 52 are right angles. The second connecting sub-sections 52 are straight lines or curves. The first affiliated sub-sections 61 and the second affiliated sub-sections 51 intersect and come into contact at the intersections 7.

In a nonrestrictive embodiment, the second connecting sub-sections 52 and/or the first connecting sub-sections 62 may be connecting points.

It should be noted that the quantity of the first affiliated sub-sections 61 and the second affiliated sub-sections 51 is related to the magnitude of the impedance between the first end 3 and the second end 4; and the quantity of the first affiliated sub-sections 61 and the second affiliated sub-sections 51 can be customized according to the actual application requirements, which is not limited in the embodiments of the present invention.

In a nonrestrictive embodiment, when the fabric structure 1 is elastically deformed, the contact area of the first affiliated sub-sections 61 and the second affiliated sub-sections 51 at the intersections 7 changes, causing the change in the impedance between the first end 3 and the second end 4. Specifically, if the area formed by contact at the intersections 7 is larger, the impedance between the first end 3 and the second end 4 is smaller.

It should be appreciated by those skilled in the art that the plurality of first affiliated sub-sections 61 and first connecting sub-sections 62 of the entry section 6 and the plurality of second affiliated sub-sections 51 and second connecting sub-sections 52 of the exit section 5 in embodiment 1 may be subjected to different sewing means from that shown in FIGS. 1 and 2, for example, they may be not straight lines.

In a nonrestrictive embodiment 2 of the present invention, as shown in FIG. 3, the wearable sensor includes an elastic yarn. The elastic yarn and a conductive yarn interweave to form a fabric structure 1. A fold-back region 2 is located within the fabric structure 1.

The conductive yarn has a first end 3, a second end 4, and a body portion (not shown). The broken line shows an entry section 8 extending from the first end 3 toward the fold-back region 2; and the solid line shows an exit section 9 returned back from the fold-back region 2.

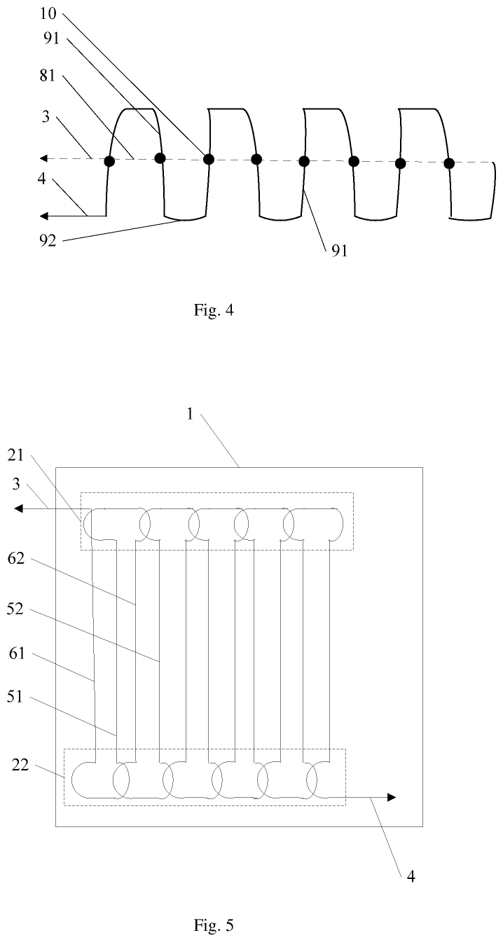

More specifically, referring to FIG. 4, the entry section 8 includes a straight section 81 extending along a straight line, and the exit section 9 includes a plurality of return sub-sections 91 and connecting sub-sections 92 connecting the adjacent return sub-sections, wherein the return sub-sections 91 and the straight section 81 form intersections 10 and come into contact at the intersections 10.

It should be appreciated by those skilled in the art that the straight section 81 may have certain curvature or be sewn in different styles.

It should also be appreciated by those skilled in the art that the designations of the entry section 8 and the exit section 9 shown in FIG. 3 can be interchanged, i.e., the section indicated by the reference numeral 8 is an exit section, and the section indicated by the reference numeral 9 is an entry section. The embodiments of the present invention do not limit the straight section 81 to be of the entry section or the exit section, or the return sub-sections 91 to be of the exit section or the entry section.

Further, the return sub-sections 91 and the straight section 81 form right angles at the intersections 10.

Understandably, when the wearable sensor is in an initial state, the return sub-sections 91 and the straight section 81 form right angles at the intersections 10. When the fabric structure 1 is deformed, the angles formed by the return sub-sections 91 and the straight section 81 at the intersections 10 are changed from right angles to non-right angles.

It should be noted that the quantity of the return sub-sections 91 is related to the magnitude of the impedance between the first end 3 and the second end 4; and the quantity of the return sub-sections 91 can be customized according to the actual application requirements, which is not limited in the embodiments of the present invention.

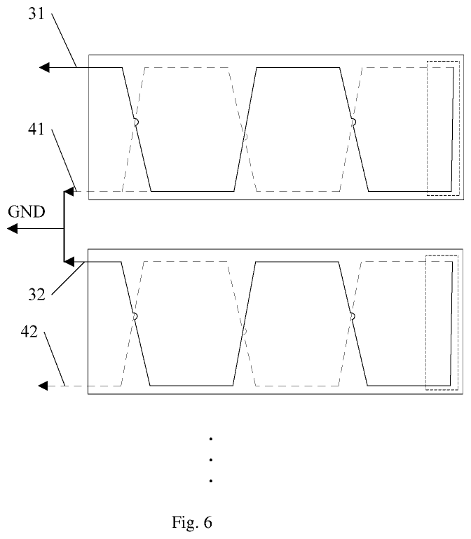

In a nonrestrictive embodiment 3 of the present invention, as shown in FIG. 5, the wearable sensor includes an elastic yarn. The elastic yarn and a conductive yarn interweave to form a fabric structure 1. A fold-back region 22 is located within the fabric structure 1.

The conductive yarn has a first end 3, a second end 4, and a body portion (not shown). An entry section 61 extends from the first end 3 toward the fold-back region 22; and an exit section 51 folds back from the fold-back region 22.

In this embodiment, the exit section 51 intersects with the entry section 61 in a secondary fold-back region 21 and then extends toward the fold-back region 22 to form a secondary entry section 62, and the secondary entry section 62 intersects with the exit section 51 in the fold-back region 22 and then folds back to form a secondary exit section 52.

The exit section 51 intersects with the entry section 61 to form an intersection, and the secondary entry section 62 intersects with the exit section 51 to form an intersection.

In this embodiment, the area formed by the contact at the intersection increases or decreases with the elastic deformation of the elastic yarn. In a nonrestrictive embodiment, when the fabric structure is deformed due to an external force, the tightness of contact at each intersection in the fold-back region changes. When the external force applied is larger, the contact at each intersection is tighter, and due to the deformation extrusion of other intersections, the contact area formed by the intersection decreases. Further, if the area formed by the intersection is smaller, the impedance between the first end 3 and the second end 4 is larger.

Further, the secondary exit section 52 intersects with the secondary entry section 62 in the secondary fold-back region 21 and then extends toward the fold-back region 22 again. By parity of reasoning, more secondary entry sections and secondary exit sections can be formed. The specific quantities of the secondary entry sections and the secondary exit sections can be customized according to the actual application scenario, and the embodiments of the present invention do not limit this.

Further, the quantity of the conductive yarn may be one or more. When the quantity of the conductive yarn is plural, the plurality of conductive yarns are twined or not twined with each other.

The conductive yarn of this embodiment can be formed by twining a plurality of conductive yarns. When the conductive yarn formed by twining interweaves with the elastic yarn to form a fabric structure 1, its contact area at the intersections increases compared to the non-twined conductive yarn, thereby increasing the linear interval of the sensor.

In a nonrestrictive embodiment, the conductive yarn is made of a metallic material. The metallic material may be stainless steel, silver, copper or other metallic materials with high conductive coefficients. The high conductivity coefficient refers to impedance less than 90-110 ohms per square centimeter, and preferably, the high conductivity coefficient refers to impedance less than 100 ohms per square centimeter.

In a nonrestrictive embodiment, the conductive yarn is made of a non-metallic material. The non-metallic material may be carbon, graphene, or the like.

In a nonrestrictive embodiment, the conductive yarn has a wrapping structure.

Further, refer to FIG. 10. The wrapping structure includes a central thread 102 and a covering thread 101 twined on the outer surface of the central thread, wherein the central thread 102 is made of a conductive material and the covering thread 101 is made of a non-conductive material, or the central thread 102 is made of a non-conductive material and the covering thread 101 is made of a conductive material.

Compared to a non-wrapping structure, the quantity of intersections is controllable or adjustable in the conductive yarn with the wrapping structure, so that the contact area of the conductive material is controllable or adjustable, and the sensitivity of the entire wearable sensor can thus be controlled or adjusted.

In addition, the two different wrapping structures are also different from each other. Specifically, in this embodiment, compared to the situation that the non-conductive material wraps the conductive material, when the conductive material wraps the non-conductive material, there are many contact points between the conductive materials, so the formed conductive area is large, and the sensing sensitivity of the sensor is high. Conversely, compared to the situation that the conductive material wraps the non-conductive material, when the non-conductive material wraps the conductive material, only a part of the conductive material is naked, and there are fewer contact points between the conductive materials, so the sensing sensitivity of the sensor is low. Therefore, the impedance value when the conductive yarns contact can be controlled by means of different wrapping structures and a wrapping density, thereby controlling the sensitivity of the sensor.

Specifically, in the conductive yarn, the conductive material made into the central thread 102, or the conductive material made into the covering thread 101, will affect the quantity of contact points between the conductive materials. That is, when the same external force is applied, compared with when the non-conductive material is made into the covering thread 101, the conductive yarn in which the conductive material is made into the covering thread 101 causes the contact points between the conductive materials increase, so the conductive area is also larger.

Experiments show that the conductive yarn with a wrapping structure has a larger linear interval, which can effectively increase the scope of application of the sensor.

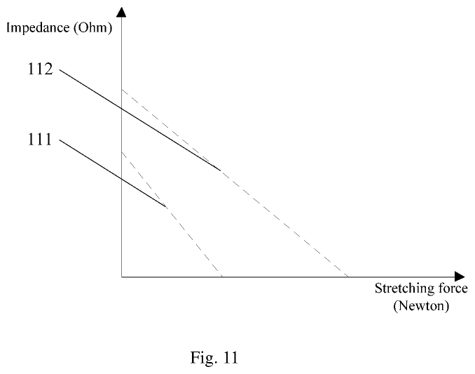

Specifically, referring to FIG. 11, in an example of a wearable sensor, the curve 111 represents, when the conductive yarn does not have a wrapping structure, the relationship between the impedance between the first end and the second end of the conductive yarn and the stretching force applied to the fabric structure. The curve 112 represents, when the conductive yarn has a wrapping structure, the relationship between the impedance between the first end and the second end of the conductive yarn and the stretching force applied to the fabric structure. It can be seen from the figure that, when the conductive yarn of the sensor has a wrapping structure, the impedance between the first end and the second end of the conductive yarn has a larger change range, and the stretching force applied to the fabric structure also has a larger change range.

Moreover, the central thread of the conductive yarn can be covered with one or more covering threads, and the resistance coefficient of the conductive yarn can be controlled by adjusting the density of the covering threads, thereby controlling the sensitivity of the sensor made of the conductive yarn according to the requirements of different usage scenarios. Furthermore, when the central thread is covered with more covering threads, the detection sensitivity of the sensor can be further improved.

In a nonrestrictive embodiment, refer to FIG. 6. A sensor module includes a plurality of sensors, taking the sensor structure shown in FIG. 1 as an example.

With regard to every two adjacent wearable sensors, such as sensor 1 and sensor 2, the conductive yarn of the sensor 1 includes a first end 31 and a second end 41; and the conductive yarn of the sensor 2 includes a first end 32 and a second end 42.

In the sensor module shown in FIG. 6, the second end 41 of the conductive yarn of one of the two adjacent sensors is connected with the first end 32 of the conductive yarn of the other sensor to form a common pole, which is grounded, for example.

One or more embodiments of the present invention further disclose a wearable sensor including a first fabric layer and a second fabric layer. The first fabric layer has at least one conductive area, the second fabric layer is attached to the first fabric layer, and the second fabric layer includes an elastic yarn and a conductive yarn. The conductive yarn and the elastic yarn interweave to form a fabric structure; the elastic yarn is made of an elastic material; the conductive yarn has a conductive capability, the conductive yarn has a first end, a second end, and a body portion between the first end and the second end, and the body portion does not have any intersection.

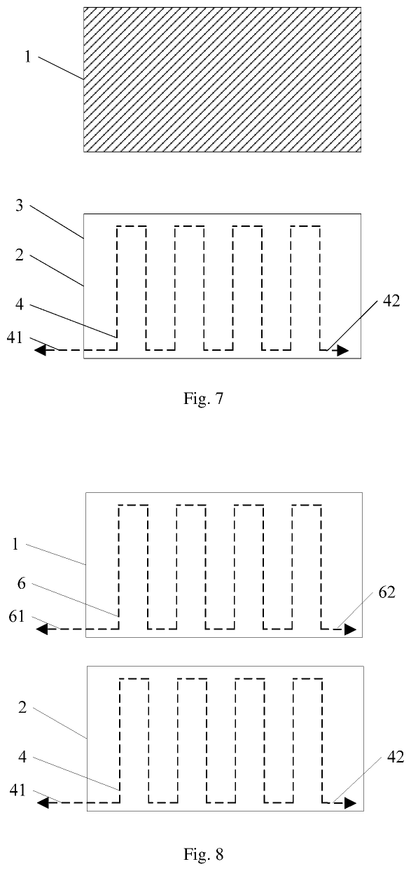

In a nonrestrictive embodiment 4 of the present invention, as shown in FIG. 7, the wearable sensor includes a first fabric layer 1 and a second fabric layer 2.

The second fabric layer 2 includes an elastic yarn and a conductive yarn 4. The elastic yarn and the conductive yarn 4 interweave to form a fabric structure 3 by a fabric weaving method. The conductive yarn 4 has a first end 41, a second end 42, and a body portion (not shown) between the first end 41 and the second end 42, and the body portion does not have any intersection.

In this embodiment, the first fabric layer 1 is a conductive fabric made of a conductive material.

In this embodiment, one of the first end 41 and the second end 42 of the conductive yarn 4 of the second fabric layer 2 can be used as a lead of the wearable sensor, which is connected together with the lead (not shown) of the first fabric layer 1 to an external electronic measuring device. When the first fabric layer 1 and the second fabric layer 2 are elastically deformed and come into contact due to the motion of a part to be detected, that is, at least a part of the body portion of the conductive yarn 4 is in contact with the first fabric layer 1, the impedance between the first end 41 and the second end 42 changes, and the motion of the part to be detected can be measured accordingly.

In this embodiment, the first fabric layer 1 can control the tension change of the elastic yarn by a fabric weaving method, and a raised fabric can be woven in the first fabric layer 1. The sensitivity of the sensor can be adjusted by means of the height of the raised fabric.

In a nonrestrictive embodiment 5 of the present invention, as shown in FIG. 8, the wearable sensor includes a first fabric layer 1 and a second fabric layer 2.

Different from the foregoing embodiment 4, the first fabric layer 1 includes the elastic yarn and the conductive yarn 6, the elastic yarn is made of an elastic material, the conductive yarn 6 has a conductive capability, and the conductive yarn 6 and the elastic yarn interweave to form a fabric structure (not shown) by a fabric weaving method.

The conductive yarn 6 has a first end 61, a second end 62, and a body portion (not shown) between the first end 61 and the second end 62, and the body portion does not have any intersection.

Further, the second end 62 of the conductive yarn 6 in the first fabric layer 1 is connected to the first end 41 of the conductive yarn 4 in the second fabric layer 2.

Further, the first end 61 of the conductive yarn 6 in the first fabric layer 1 and the first end 41 of the conductive yarn 4 in the second fabric layer 2 can be used as two leads of the wearable sensor, which are connected to an external electronic measuring device. When the first fabric layer 1 and the second fabric layer 2 are elastically deformed and come into contact due to the motion of a part to be detected, that is, the body portion of the conductive yarn 6 is in contact with at least a part of the body portion of the conductive yarn 4, the impedance between the first end 61 of the conductive yarn 6 in the first fabric layer 1 and the first end 41 of the conductive yarn 4 in the second fabric layer 2 changes, and the motion of the part to be detected can be measured accordingly.

Understandably, the second end 62 of the conductive yarn 6 in the first fabric layer 1 and the second end 42 of the conductive yarn 4 in the second fabric layer 2 can be used as two leads of the wearable sensor for external connection; or, the first end 61 of the conductive yarn 6 in the first fabric layer 1 and the second end 42 of the conductive yarn 4 in the second fabric layer 2 can be used as two leads of the wearable sensor for external connection; or the second end 62 of the conductive yarn 6 in the first fabric layer 1 and the first end 41 of the conductive yarn 4 in the second fabric layer 2 can be used as two leads of the wearable sensor for external connection. The embodiments of the present invention do not limit this.

Further, in this embodiment, the body portion of the conductive yarn in the first fabric layer 1 and the body portion of the conductive yarn in the second fabric layer 2 are not limited in this embodiment, and they can be perpendicular or parallel to each other.

Further, in the above-mentioned embodiment 4 and embodiment 5, the body portion may include a plurality of U-shaped sections and connecting sections connecting the adjacent U-shaped sections. In addition, it should be appreciated by those skilled in the art that the body portion may not include U-shaped sections, but is in other shapes without intersections, such as a triangle or a trapezoid.

One or more embodiments of the present invention further disclose a sensor module, wherein in every two adjacent wearable sensors, the second end of the conductive yarn of the second fabric layer of one wearable sensor is connected to the first end of the conductive yarn of the second fabric layer of the other wearable sensor.

The wearable sensor disclosed in one or more embodiments of the present invention has wide application fields. If the pressure applied to the sensor is measured by means of the impedance change characteristics described above, the sensor can be used for the detection of breathing, joint motion, limb motion, and bed leaving; by means of the electrical conductivity of the conductive yarn, the sensor can be used as a conductive electrode for relevant sensing of ECG, heartbeat, myoelectricity, low-frequency electrotherapy, and the like; if the conductive yarn is made of a metallic material, the sensor can be used as a temperature sensor to measure temperature or as a cooling fabric product by means of the thermal conductivity of metal; and by means of the impedance of the conductive yarn, a DC PWM voltage can be applied to the conductive yarn to control the magnitude of the voltage, and the sensor can be used as a controllable heating device. Therefore, the wearable sensor disclosed in one or more embodiments of the present invention can be used as a multifunctional composite sensor.

Further, if the wearable sensor disclosed in one or more embodiments of the present invention is applied to a foot pad or a glove, the wearable sensor can be used as a sensor for body sensing by means of the flexibility, electrical conductivity and stretching impedance change of the sensor of the present invention, to precisely detect body motion and then to record the motion status of the wearer, so that the wearable sensor can be applied to interactive games or human rehabilitation training. At present, common limb sensing usually employs a camera for image recognition, which can only recognize a human gesture by a large margin, but cannot detect whether the motion is accurate, and the image recognition requires certain spatial range for clear recognition, whereas the wearable sensor disclosed in one or more embodiments of the present invention does not have these disadvantages.

Further, the sensor module disclosed in one or more embodiments of the present invention includes a plurality of sensors, so that the sensor module can cover a large area of the human body for large-area motion detection.

In a typical application scenario of the present invention, the sensor of the present invention can be fixed to the crotch of trousers. When the trousers worn by the user are deformed by stretching, the sensor can detect human motion of the crotch. For example, when there is only one sensor, the sensor can monitor erection; and when the sensor module is used, the sensor module can detect not only erection, but also the direction of erection.

In another typical application scenario of the present invention, the pressure applied to the sensor is measured by means of the impedance change characteristics described above, which can be used for the detection of bed leaving. By detecting whether the user leaves the bed, it can be determined whether the user falls.

Although the present invention is disclosed above, the present invention is not limited thereto. Any person skilled in the art can make various changes and modifications without departing from the spirit and scope of the present invention. Therefore, the protection scope of the present invention should be subject to the scope of the claims.

1. A wearable sensor, comprising:
an elastic yarn made of an elastic material; and
a conductive yarn;
wherein the conductive yarn and the elastic yarn interweave and form a fabric structure;
wherein the conductive yarn has a first end, a second end, and a body portion between the first end and the second end, the body portion comprises an entry section extending from the first end toward a fold-back region and an exit section returned back from the fold-back region, the entry section and the exit section form at least one intersection, and the entry section and the exit section come into contact at the intersection.

2. The sensor according to claim 1, wherein an area formed by the contact of the entry section and the exit section at the intersection changes with an elastic deformation of the elastic yarn.

3. The sensor according to claim 2, wherein, when the fabric structure is deformed due to an external force, the area formed by the contact at the intersection increases with the elastic deformation of the elastic yarn.

4. The sensor according to claim 2, wherein, when the fabric structure is deformed due to an external force, the area formed by the contact at the intersection decreases with the elastic deformation of the elastic yarn.

5. (canceled)

6. The sensor according to claim 1, wherein the entry section comprises a plurality of adjacent first sub-sections; the exit section comprises a plurality of adjacent second sub-sections; wherein the plurality of adjacent first sub-sections and the plurality of adjacent second sub-sections intersect and come into contact at the intersections.

7. The sensor according to claim 1, wherein one of the entry section and the exit section comprises a straight section extending along a straight line, and the other one of the entry section and the exit section comprises a plurality of return sub-sections and connecting sub-sections connecting the return sub-sections; wherein the return sub-sections and the straight section form intersections and contact each other at intersections.

8. The sensor according to claim 1, wherein the conductive yarn and the elastic yarn interweave by a fabric weaving method; wherein the exit section intersects with the entry section in a secondary fold-back region and then extends toward the fold-back region to form a secondary entry section, and the secondary entry section intersects with the exit section in the fold-back region and then folds back to form a secondary exit section.

9. The sensor according to claim 8, wherein the secondary exit section intersects with the secondary entry section in the secondary fold-back region and then extends toward the fold-back region again.

10.-12. (canceled)

13. A forming method of a wearable sensor, comprising:
providing an elastic yarn made of an elastic material;
providing a conductive yarn having a first end, a second end, and a body portion between the first end and the second end; and
interweaving the conductive yarn and the elastic yarn and forming a fabric structure,
wherein the fabric structure has a fold-back region, a part of the body portion that extends from the first end toward the fold-back region is used as an entry section, a part returned back from the fold-back region is used as an exit section, the entry section and the exit section form at least one intersection, and the entry section and the exit section come into contact at the intersection.

14. The forming method according to claim 13, wherein an area formed by the contact of the entry section and the exit section at the intersection changes with an elastic deformation of the elastic yarn.

15. The forming method according to claim 14, wherein, when the fabric structure is deformed due to an external force, the area formed by the contact at the intersection increases with the elastic deformation of the elastic yarn.

16. The forming method according to claim 14, wherein, when the fabric structure is deformed due to an external force, the area formed by the contact at the intersection decreases with the elastic deformation of the elastic yarn.

17. (canceled)

18. The forming method according to claim 13, wherein the entry section comprises a plurality of adjacent first sub-sections; the exit section comprises a plurality of adjacent second sub-sections; and interweaving the entry section and the exit section with the elastic yarn to form at least one intersection comprises:
making the plurality of adjacent first sub-sections and the plurality of adjacent second sub-sections interweave with the elastic yarn and come into contact at intersections.

19. The forming method according to claim 13, wherein one of the entry section and the exit section comprises a straight section extending along a straight line, and the other one comprises a plurality of return sub-sections and connecting sub-sections connecting the return sub-sections; and the return sub-sections, the straight section and the elastic yarn interweave to form intersections and come into contact at the intersections.

20. The forming method according to claim 13, wherein the conductive yarn and the elastic yarn interweave by a fabric weaving method; the exit section intersects with the entry section in a secondary fold-back region and then extends toward the fold-back region to form a secondary entry section; and the secondary entry section intersects with the exit section in the fold-back region and then folds back to form a secondary exit section.

21. The forming method according to claim 20, wherein the secondary exit section intersects with the secondary entry section in the secondary fold-back region and then extends toward the fold-back region again.

22.-26. (canceled)

27. A wearable sensor, comprising:
a first fabric layer having at least one conductive area; and
a second fabric layer attached to the first fabric layer, the second fabric layer comprising:
an elastic yarn made of an elastic material; and
a conductive yarn having at least a wrapping structure;
wherein the conductive yarn and the elastic yarn interweave and form a fabric structure; wherein the conductive yarn has a first end, a second end, and a body portion between the first end and the second end, and the body portion does not have any intersection.

28. The sensor according to claim 27, wherein when the fabric structure is deformed due to an external force, a contact area of the second fabric layer and the first fabric layer changes with an elastic deformation of the elastic yarn.

29. The sensor according to claim 27, wherein the wrapping structure comprises a central thread and a covering thread twined outside the central thread; wherein the central thread is made of a non-conductive material and the covering thread is made of a conductive material.

30. The sensor according to claim 27, wherein the body portion comprises a plurality of adjacent U-shaped connecting sections.

31. The sensor according to claim 27, wherein the first fabric layer comprises the elastic yarn and the conductive yarn, and the elastic yarn is made of an elastic material; the conductive yarn has at least a wrapping structure; the conductive yarn and the elastic yarn interweave to form a fabric structure; wherein the conductive yarn has a first end, a second end, and a body portion between the first end and the second end, and the body portion does not have any intersection.

32. (canceled)

33. The sensor according to claim 27, wherein the first fabric layer comprises a raised fabric portion, which is formed by controlling interweaving tension of the elastic yarn in the first fabric layer.

34. A forming method of a wearable sensor, comprising:
providing a first fabric layer having at least one conductive area;
providing a second fabric layer attached to the first fabric layer, the second fabric layer comprising an elastic yarn made of an elastic material and a conductive yarn having at least a wrapping structure; and
interweaving the conductive yarn and the elastic yarn in the second fabric layer and forming a fabric structure, wherein the conductive yarn has a first end, a second end, and a body portion between the first end and the second end, and the body portion does not have any intersection.

35. The forming method according to claim 34, wherein, when the fabric structure is deformed due to an external force, a contact area of the second fabric layer and the first fabric layer changes with an elastic deformation of the elastic yarn.

36.-40. (canceled)

41. The sensor according to claim 1,
wherein the conductive yarn has a wrapping structure;
wherein the wrapping structure comprises a central thread and a covering thread twined outside the central thread; and
wherein the central thread is made of a conductive material and the covering thread is made of a non-conductive material, or the central thread is made of a non-conductive material and the covering thread is made of a conductive material.

42. The forming method according to claim 13,
wherein the conductive yarn has a wrapping structure.
wherein the wrapping structure comprises a central thread and a covering thread twined outside the central thread; and
wherein the central thread is made of a conductive material and the covering thread is made of a non-conductive material, or the central thread is made of a non-conductive material and the covering thread is made of a conductive material.
